(19) United States
(12) Patent Application Publication
(10) Pub. No.: US 2014/0161755 A1
(43) Pub. Date: Jun. 12, 2014

(54) CLEAR CLEANSING COMPOSITION

(71) Applicant: L'OREAL, Paris (FR)

(72) Inventors: Shilpa ARORA, CARTERET, NJ (US); Marie HUYNH, MONMOUTH JUNCTION, NJ (US); Carmen CASTILLO, ENGLEWOOD, NJ (US)

(73) Assignee: L'OREAL, Paris (FR)

(21) Appl. No.: 13/709,544

(22) Filed: Dec. 10, 2012

(51) Int. Cl. A61K 8/04; A61Q 5/12; A61Q 5/02

(52) U.S. Cl. CPC A61K 8/044; A61Q 5/02; A61Q 5/12
USPC 424/70.12

(57) ABSTRACT

The present invention is directed to a cleansing composition containing: (a) at least one anionic surfactant; (b) at least one amphoteric surfactant; (c) at least one amphoteric polymer; (d) at least one acrylate copolymer; (e) at least one water-soluble silicone; (f) at least one alcohol ester, and wherein the composition is clear in appearance and free of cationic polymer.

### BACKGROUND OF THE INVENTION

The present invention relates to personal cleansing compositions. More particularly, the invention relates to cleansing compositions that provide exceptional cleansing and conditioning properties on keratinous substrates.

Conventional cleansing compositions such as shampoos, for example, contain standard surfactants such as anionic, nonionic and/or amphoteric type surfactants. These compositions can be applied onto a wet keratinous substrate and the lather they generate make it possible, after rinsing with water, to remove the diverse types of soils typically present on the substrate such as hair and/or skin.

These cleansing compositions, while providing good cleansing power, may yield poor intrinsic cosmetic properties due to the fact that the relatively aggressive nature of such a cleansing treatment may, in the long term, give rise to more or less pronounced damage on hair fibers associated, for example, with the gradual removal of the fats or proteins contained in or at their surface.

Thus, in order to improve the cosmetic properties of cleansing compositions, it is now common practice to introduce into these compositions cationic polymers for use as conditioning agents in order to improve the tactile properties of said compositions. The use of cationic polymers, however, oftentimes has a limitation on foam quality in terms of volume of foam generated and sensorial feel of the foam. Moreover, the use of cationic polymers typically results in the keratinous substrate, such as hair for example, feeling weighted and heavy.

It is thus an object of the present invention to provide a cleansing composition which cleans and conditions a keratinous substrate leaving it silky, soft and weightless feeling, and while at the same time generating voluminous, luxurious feeling foam.

It is also an object of the present invention to provide such a cleansing composition which is clear in appearance.

### BRIEF SUMMARY OF THE INVENTION

The present invention is directed to a cleansing composition containing:

(a) at least one anionic surfactant;

(b) at least one amphoteric surfactant;

(c) at least one amphoteric polymer;

(d) at least one acrylate copolymer;

(e) at least one silicone;

(f) at least one alcohol ester, and

wherein the composition is preferably clear in appearance and free of cationic polymer.

The present invention is also directed to a process for cleansing and conditioning a keratinous substrate involving contacting the keratinous substrate with the above-disclosed cleansing composition.

It has been surprisingly discovered that the above-described composition provides not only good cleansing of keratinous substrates, but does so in a cosmetically elegant way in terms of volume and luxuriousness of foam generated by the composition, while at the same time imparting conditioning properties onto the substrate in the absence of cationic polymers.

It is to be understood that both the foregoing general description and the following detailed description are exemplary and explanatory only, and are not restrictive of the invention.

### DETAILED DESCRIPTION OF THE INVENTION

Other than in the operating examples, or where otherwise indicated, all numbers expressing quantities of ingredients and/or reaction conditions are to be understood as being modified in all instances by the term “about”, meaning within 10% to 15% of the indicated number.

“Keratinous substrates”, as used herein, include but are not limited to, skin, hair, lips, eyelashes and nails.

As used herein, the expression “at least one” means one or more and thus includes individual components as well as mixtures/combinations.

As used herein, the expression “free of” means that an ingredient may, but need not, be present in the composition.

As used herein, the term “clear” means that the composition allows a transmittance of light at 600 nm of greater than 85%, such as greater than 90% and further such as greater than 94%.

### Anionic Surfactant

The at least one anionic surfactant used in the cosmetic compositions disclosed herein can be, for example, chosen from salts, for example, alkali metal salts such as sodium salts, ammonium salts, amine salts, amino alcohol salts and alkaline-earth metal salts, for example magnesium salts, of the following types: alkyl sulfates, alkyl ether sulfates, alkylamido ether sulfates, alkylaryl polyether sulfates, monoglyceride sulfates, alkylsulfonates, alkylamide sulfonates, alkylarylsulfonates, .alpha.-olefin sulfonates, paraffin sulfonates, alkylsulfosuccinates, alkyl ether sulfosuccinates, alkylamide sulfosuccinates, alkylsulfoacetates, acylsarcosinates and acylglutamates, the alkyl and acyl groups of all these compounds comprising from 6 to 24 carbon atoms and the aryl group, for example, representing a phenyl or benzyl group.

It is also possible to use C6-24 alkyl monoesters of polyglycoside-dicarboxylic acids such as alkyl glucoside citrates, alkyl polyglycoside tartrates and alkyl polyglycoside sulfosuccinates, alkylsulfosuccinamates, acylisethionates and N-acyltaurates, the alkyl or acyl group of all these compounds comprising from 12 to 20 carbon atoms.

Another group of anionic surfactants that may be used in the compositions disclosed herein is that of acyllactylates in which the acyl group comprises from 8 to 20 carbon atoms.

In addition, exemplary mention may also be made of alkyl-D-galactoside uronic acids and salts thereof and also polyoxyalkylenated (C6-24 alkyl)ether carboxylic acids, polyoxyalkylenated (C6-24 alkyl)(C6-24 aryl)ether carboxylic acids, polyoxyalkylenated (C6-24 alkyl)amido ether carboxylic acids and salts thereof, such as those comprising from 2 to 50, further such as from 2 to 10 and even further such as from 2 to 5 ethylene oxide units, and mixtures thereof.

According to at least one embodiment, the at least one anionic surfactant is chosen from alkyl sulfates and alkyl ether sulfates, for example in the form of alkali metal, alkaline-earth metal, ammonium, amine and amino alcohol salts.

According to at least one embodiment, the at least one anionic surfactant used in the composition disclosed herein can be chosen from alkyl ether sulfates, such as C12-C14 and for example, comprising from 2 to 3 mol of ethylene oxide, and N-acyltaurates.

The at least one anionic surfactant is present in a total amount ranging from about 3% to about 30% by weight, such as in a total amount ranging from about 3% to about 25% by weight and further such as in a total amount ranging from about 3% to about 15% by weight relative to the total weight of the cleansing composition disclosed herein.

### Amphoteric Surfactant

The amphoteric or zwitterionic surfactants that may be used in the present disclosure may, for example, be secondary or tertiary aliphatic amine derivatives in which the aliphatic group is a linear or branched chain comprising from 8 to 22 carbon atoms and comprising at least one anionic group, for instance a carboxylate, sulfonate, sulfate, phosphate or phosphonate group. Mention may be made, for example, of (C8-C20)alkylbetaines, sulfobetaines, (C8-C20)alkylamido(C6-C8)alkylbetaines or (C8-C20)alkylamido(C6-C8)alkylsulfobetaines.

Among the amine derivatives, exemplary mention may be made of the products sold under the name MIRANOL®, as described, for example, in U.S. Pat. No. 2,528,378 and U.S. Pat. No. 2,781,354 and classified in the CTFA dictionary, 3rd edition, 1982, under the names Amphocarboxyglycinate and Amphocarboxypropionate, having the respective structures (IV) and (V):

Ra-CONHCH2CH2-N(Rb)(Rc)(CH2COO (IV)

in which:

Ra represents the alkyl group from an acid Ra-COOH present in hydrolysed coconut oil, a heptyl, nonyl or undecyl group,

Rb represents a beta-hydroxyethyl group, and

Rc represents a carboxymethyl group; and

Ra′-CONHCH2CH2-N(B)(B′) (V)

in which:

B represents —CH2CH2OX′,

B′ represents —(CH2).sub.z—Y′, with z=1 or 2,

X′ represents —CH2CH2-COOH or hydrogen,

Y′ represents —COOH or —CH2-CHOH—SO3H,

Ra′ represents the alkyl group of an acid Ra′-COOH present in coconut oil or in hydrolysed linseed oil, an alkyl group, for example, a C1-7 alkyl group and its iso form, or an unsaturated C17 group.

These compounds can be classified in the CTFA dictionary, 5th edition, 1993, under the names disodium cocoamphodiacetate, disodium lauroamphodiacetate, disodium caprylamphodiacetate, disodium caprylamphodiacetate, disodium cocoamphodipropionate, disodium lauroamphodipropionate, disodium caprylampho-dipropionate, disodium caprylamphodipropionate, lauroamphodipropionic acid, or cocoamphodipropionic acid.

By way of example, mention may be made of the cocoamphodiacetate sold by the company Rhodia under the trade name MIRANOL® C2M Concentrate.

The at least one amphoteric or zwitterionic surfactant may be, for example, chosen from (C8-20 alkyl) betaines and (C8-20 alkyl)amido(C6-8 alkyl)betaines.

The at least one amphoteric or zwitterionic surfactant is present in a total amount ranging from about 1% to about 40% by weight and further such as in a total amount ranging from about 1% to about 15% by weight relative to the total weight of the cleansing composition disclosed herein.

### Amphoteric Polymer

In general, polymeric quaternary ammonium salts are cationic or amphoteric polymers comprising at least one quaternized nitrogen atom. However, in the present invention, these polymers are amphoteric by virtue of the fact that the compositions of the present invention have a pH of at least about 5.

Mention may in particular be made, as polymeric quaternary ammonium salts, of the Polyquaternium products (CTFA name), which contribute softness and smoothness to the foaming cream. These polymers can preferably be chosen from the following:

Polyquaternium-39, such as the product Merquat Plus 3330, Merquat Plus 3331, Merquat 3333 sold by Nalco (Lubrizol);

Polyquaternium-47, such as the product Merquat 2001 sold by Nalco (Lubrizol).

The at least one amphoteric polymer is present in a total amount ranging from about 0.01% to about 5% by weight and further such as in a total amount ranging from about 0.01% to about 2% by weight relative to the total weight of the cleansing composition disclosed herein.

### Acrylates Copolymer

The composition of the present invention further contains at least one crosslinked copolymer containing at least one monomer selected from methacrylic acid, acrylic acid, amino acrylic acid, an acrylic acid ester of a C8-C30 alkyl, alkyaryl, aryl, heterocyclic, alkoxyl, alkoxyl alkyl ester of a C8-C30 alkyl or alkenyl; which may be substituted; a methacrylic acid ester of a C8-C30 alkyl, alkylaryl, aryl, heterocyclic, alkoxyl, alkoxyl alkyl ester of a C8-C30 alky, or alkenyl; which may be substituted; a C1-C4 alkyl acrylate and a C1-C4 methacrylate; which may be substituted.

Particular mention may be made of a crosslinked polymer comprising at least one methacrylic acid unit and at least one C1-C4 alkyl acrylate unit. The crosslinked copolymer comprising at least one methacrylic acid unit and at least one C1-C4 alkyl acrylate unit means a crossklinked copolymer comprised of at least one methacrylic acid unit and at least one alkyl acrylate unit, wherein the alkyl acrylate is chosen from C1-C4 alkyl acrylates.

In the crosslinked copolymer disclosed herein, the at least one methacrylic acid unit can be present in an amount ranging from 20% to 80% by weight, for example, from 25% to 70% by weight, and further, for example, from 35% to 60% by weight, relative to the total weight of the copolymer.

In the crosslinked copolymer disclosed herein, the at least one alkyl acrylate unit can be present in an amount ranging from 15% to 80% by weight, for example, from 25% to 75% by weight, and further, for example, from 40% to 65% by weight, relative to the total weight of the copolymer. The alkyl acrylate can, for example, be chosen from methyl acrylate, ethyl acrylate and butyl acrylate. In one embodiment, the alkyl acrylate is ethyl acrylate.

The copolymer disclosed herein is partially or totally crosslinked with at least one crosslinking agent. The at least one crosslinking agent can be chosen, for example, from polyunsaturated compounds, such as polyethylenically unsaturated compounds. These compounds can be chosen, for example, from polyalkenyl ethers of sucrose, polyalkenyl ethers of polyols, diallyl phthalates, divinylbenzene, allyl(meth)acrylate, ethylene glycol di(meth)acrylate, methylenebisacrylamide, trimethylolpropane tri(meth)acrylate, diallyl itaconate, diallyl fumarate, diallyl maleate, zinc(meth)acrylate, castor oil derivatives and polyol derivatives manufactured from unsaturated carboxylic acids.

The at least one crosslinking agent that may also be used include, for example, unsaturated monomers comprising at least one reactive group capable of reacting with an unsaturation to form a crosslinked copolymer.

The content of the at least one crosslinking agent generally ranges from 0.01% to 5% by weight, for example, from 0.03% to 3% by weight, and further, for example, from 0.05% to 1% by weight, relative to the total weight of the copolymer.

According to one embodiment, the at least one crosslinked copolymer disclosed herein may, for example, be in the form of a dispersion in water. The number-average size of the copolymer particles in the dispersion generally ranges from 10 nm to 500 nm, for example, from 20 nm to 200 nm, and further, for example, from 50 nm to 150 nm.

These copolymers are described, for example, in Patent Application No. WO 01/76552.

For example, the crosslinked methacrylic acid/ethyl acrylate copolymer in the form of an aqueous dispersion of 30% active material manufactured and sold under the name Carbopol Aqua SF-1 by the company Noveon may be used.

The at least one acrylates copolymer is present in a total amount ranging from about 0.15% to about 10% by weight and further such as in a total amount ranging from about 0.15% to about 5% by weight relative to the total weight of the cleansing composition disclosed herein.

### Silicone

Suitable silicones include those modified with oxyalkylene groups. Monovalent oxyalkylenated groups, are preferably chosen, for example, from the compounds of general formulae (I), (II), (III) and (IV):

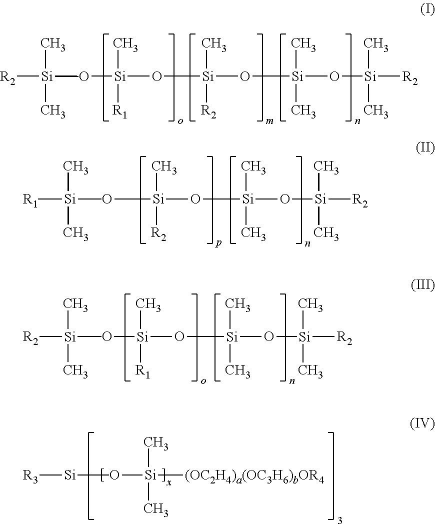

in which formulae:

R1 represents a phenyl or C1-C30 alkyl radical,

R2 represents (CH2)c(-C2 H4 O)a(-C3 H6 O)b-R5,

R3 and R4, which may be identical or different, denote a C1-C12 alkyl radical,

R5 is chosen from a hydrogen atom, an alkyl radical of 1 to 12 carbon atoms, an alkoxy radical of 1 to 6 carbon atoms, a hydroxyl radical, —SO3 M, —OCOR6, C1-C6 aminoalkoxy optionally substituted on the amine, C1-C6 aminoacyl optionally substituted on the amine, —NHCH2 CH2 COOM, N(CH2 CH2 COOM)2, aminoalkyl, preferably containing from one to six carbon atoms, optionally substituted on the amine and on the alkyl chain, C1-C30 carboxyacyl, a phosphono group optionally substituted with one or two substituted aminoalkyl radicals, —CO(CH2)d COOM, —OCOCHR7 (CH2)d COOM, —NHCO(CH2)d OH and NH3 Y,

M denotes a hydrogen atom, Na, K, Li, NH4 or an organic amine,

R6 denotes a C1-C30 alkyl radical,

R7 denotes hydrogen or SO3 M, d ranges from 1 to 10, m ranges from 0 to 20, n ranges from 1 to 100, o ranges from 0 to 20, p ranges from 1 to 20, a ranges from 0 to 50, b ranges from 0 to 50, a+b is greater than or equal to 1, c ranges from 0 to 4, x ranges from 1 to 100,

Y represents a monovalent inorganic or monovalent organic anion.

Silicones corresponding to the general formula (I) or (II) are preferably used, and in particular those in which R5 denotes a hydrogen atom.

Preferably, c is equal to 2 or 3.

Such silicones are sold, for example, by the company Goldschmidt under the trade names ABIL WE 09, ABIL EM 90, ABIL B8852, ABIL B8851, ABIL B8843 and ABIL B8842, by the company Dow Corning under the names FLUID DC 190, DOW CORNING 193, DC 3225 C, Q2-5220, Q2-5354 and Q2-5200, by the company Rhone-Poulenc under the names SILBIONE OIL 70646 and RHODORSIL OIL 10634, by the company General Electric under the names SF1066 and SF1188, by the company SWS Silicones under the name SILICONE COPOLYMER F 754, by the company Amerchol under the name SILSOFT BEAUTY AID SL, by the company Shin-Etsu under the name KF 351, by the company Wacker under the name BELSIL DMC 6038, by the company Siltech under the names SILWAX WD-C, SILWAX WD-B, SILWAX WD-IS, SILWAX WS-L, SILWAX DCA 100 and SILTECH AMINE 65, and by the company Fanning Corporation under the names FANCORSIL SLA and FANCORSIL LIM1.

The at least one silicone is present in a total amount ranging from about 0.01% to about 8% by weight and further such as in a total amount ranging from about 0.01% to about 4% by weight relative to the total weight of the cleansing composition disclosed herein.

### Alcohol Ester

Suitable alcohol esters for use in the present invention include alkyl benzoates, isopropyl palmitate, isopropyl myristate, diisopropyl adipate, and derivates thereof. More particularly, the esters may be chosen from: hydrocarbon-based esters of formula RCOOR′ in which RCOO represents a carboxylic acid residue comprising from 2 to 40 carbon atoms, and R′ represents a hydrocarbon-based chain containing from 1 to 40 carbon atoms, such as cetostearyl octanoate, isopropyl alcohol esters, such as isopropyl myristate or isopropyl palmitate, ethyl palmitate, 2-ethylhexyl palmitate, isopropyl stearate or isostearate, isostearyl isostearate, octyl stearate, diisopropyl adipate, heptanoates, and especially isostearyl heptanoate, alcohol or polyalcohol octanoates, decanoates or ricinoleates, for instance propylene glycol dioctanoate, cetyl octanoate, trioleoyl octanoate, 2-ethylhexyl 4-diheptanoate and palmitate, alkyl benzoate, polyethylene glycol diheptanoate, propylene glycol 2-diethyl hexanoate, and mixtures thereof, C12 to C15 alcohol benzoates, hexyl laurate, neopentanoic acid esters, for instance isodecyl neopentanoate, isotridecyl neopentanoate, isostearyl neopentanoate and 2-octyldodecyl neopentanoate, isononanoic acid esters, for instance isononyl isononanoate, isotridecyl isononanoate and octyl isononanoate, oleyl erucate, isopropyl lauroyl sarcosinate, diisopropyl sebacate, isocetyl stearate, isodecyl neopentanoate, isostearyl behenate, and myristyl myristate.

The at least one alcohol ester is present in a total amount ranging from about 0.01% to about 7% by weight and further such as in a total amount ranging from about 0.01% to about 4% by weight relative to the total weight of the cleansing composition disclosed herein.

### Optional Nonionic Agent

The composition of the present invention may also contain a nonionic agent such as a nonionic surfactant and/or a nonionic polymer.

Suitable nonionic polymers include fatty acid amides such as, for example, coconut monoethanolamide, coconut diethanolamide, oxyethylenated carboxylic acid alkyl ether monoethanolamide; cellulose-based thickeners such as hydroxymethylcellulose, hydroxyethylcellulose, hydroxypropylcellulose and hydroxypropylmethylcellulose; guar gum and derivatives thereof such as hydroxypropyl guar gum; gums of microbial origin such as xanthan gum and cleroglucan gum; and nonionic associated polymers.

Suitable nonionic surfactants include polyethoxylated, polypropoxylated and polyglycerolated fatty acids, (C1-C0)alkylphenols, .alpha.-diols and alcohols, having a fatty chain comprising, for example, 8 to 18 carbon atoms, the number of ethylene oxide or propylene oxide groups possibly ranging for example from 2 to 50 and the number of glycerol groups possibly ranging for example from 2 to 30.

Mention may also be made, for example, of condensates of ethylene oxide and/or of propylene oxide with fatty alcohols; polyethoxylated fatty amides for example having from 2 to 30 ethylene oxide units, polyglycerolated fatty amides comprising on average 1 to 5, and for example from 1.5 to 4, glycerol groups, ethoxylated fatty acid esters of sorbitan comprising from 2 to 30 ethylene oxide units, fatty acid esters of sucrose, fatty acid esters of polyethylene glycol, (C6-24 alkyl)polyglycosides, N—(C6-24 alkyl)glucamine derivatives, amine oxides such as (C10-C14)alkylamine oxides or N—(C10-14 acyl)aminopropylmorpholine oxides.

The at least one nonionic agent may be present in a total amount ranging from about 0.1% to about 10% by weight and further such as in a total amount ranging from about 0.1% to about 5% by weight relative to the total weight of the cleansing composition disclosed herein.

According to a particularly preferred embodiment of the present invention, the cleansing composition is clear in appearance and free of cationic polymers.

According to a preferred embodiment of the present invention, cleansing compositions capable of imparting improved cosmetic properties onto a keratinous substrate cleansed therewith such as, for example, silkiness, softness and weightlessness, while at the same time generating voluminous, luxurious feeling foam, are provided.

The present invention is also directed to a process for cleansing and conditioning a keratinous substrate involving contacting the keratinous substrate with the above-disclosed cleansing composition.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

| | Ex. 1 | Ex. 2 | Ex. 3 | Ex. 4 | Ex. 5 | Ex. 6 |
|---|---|---|---|---|---|---|
| INCI US | % wt/wt | % wt/wt | % wt/wt | % wt/wt | % wt/wt | % wt/wt |
| SODIUM LAURETH | 14.7 | 14.7 | 11.9 | 11.9 | 11.9 | 11.9 |
| SULFATE | | | | | | |
| COCO-BETAINE | 3 | 3 | 3 | 3 | 3 | 3 |
| POLYQUATERNIUM-39 | | | | 0.185 | 0.185 | 0.185 |
| ACRYLATES | | 2.4 | 3 | 3 | 3 | 3 |
| COPOLYMER | | | | | | |
| PEG-14 | | | 0.7 | 0.7 | | 0.7 |
| DIMETHICONE | | | | | | |
| AMODIMETHICONE | | 0.8 | | | 0.8 | |
| ISOPROPYL | | | | | | 1 |
| PALMITATE | | | | | | |
| PEG-30 GLYCERYL | 1 | 1 | 1 | 1 | 1 | 1 |
| COCOATE | | | | | | |
| WATER | Q.S. | Q.S. | Q.S. | Q.S. | Q.S. | Q.S. |
| | to 100 | to 100 | to 100 | to 100 | to 100 | to 100 |
| Swatch testing evaluated | Poor | Good | Good | Good | Good | Excellent |
| by Industrial expert (foam, | | | | | | |
| smooth, softness, style . . .) | | | | | | |

*All of the numbers in the above table are percent by weight active, based on the total weight of the compostion

What is claimed is:

1. (canceled)

2. (canceled)

3. The composition of claim 17 wherein (a) is chosen from Sodium Laureth Sulfate, Sodium Lauryl Sulfate, Ammonium Lauryl Sulfate, Ammonium Laureth Sulfate, Sodium Cocoyl Isethionate, Sodium Lauroyl Methyl Isethionate, Tea-Cocoyl Glutamate, Sodium Lauroyl Glutamate, Disodium Cocoyl Glutamate, Sodium Cocoyl Glutamate, Sodium Cocoyl Alaninate, Sodium cocoyl Glycinate, Disodium Laureth Sulfosuccinate, Sodium Lauryl Sulfoacetate, Disodium Laureth Sulfosuccinate, Lauryl Phosphate, Potassium Phosphate, Sodium Methyl Cocoyl Taurate, Sodium Cocoyl Amino Acids, Sodium Cocoyl Apple Amino Acids, Sodium Lauroyl Sarcosinate, Sodium Cocoyl Sarcosinate, Lauryl Glucoside Sodium Lauryl Glucose Carboxylate, and mixtures thereof.

4. (canceled)

5. The composition of claim 3 wherein (b) is chosen from Coco Betaine, Cocamidopropyl Betaine, glyceryl laurate capryl/capramidopropyl betaine, Lauryl betaine, Cocamidopropyl hydroxysultaine, sodium cocoamphodiacetate, sodium cocoamphoacetate, sodium cocoamphopropionate, and mixtures thereof.

6. (canceled)

7. The composition of claim 5 wherein (c) is chosen from polyquaternium-39, polyquaternium-47, and mixtures thereof.

8. (canceled)

9. The composition of claim 7 wherein (d) is chosen from acrylates copolymer, polyacrylate-33, acrylates/vinyl neodecanoate crosspolymer, acrylates/steareth-20 methacrylate crosspolymer, and mixtures thereof.

10. (canceled)

11. The composition of claim 9 wherein (e) is chosen from PEG-14 Dimethicone, PEG/PPG-14/4 Dimethicone, PEG/PPG-4/12 Dimethicone, Cetyl PEG/PPG 10/1 Dimethicone, amodimethicone, dimethiconol, phenyltrimethicone, and mixtures thereof.

12. (canceled)

13. The composition of claim 11 wherein (f) is chosen from Isopropyl Palmitate, Isopropyl Myristate, Myristyl Myristate, and mixtures thereof.

14. (canceled)

15. (canceled)

16. (canceled)

17. A cleansing and conditioning composition comprising:
(a) from about 3% to about 30% by weight of anionic surfactant;
(b) from about 1% to about 15% by weight of amphoteric surfactant;
(c) from about 0.01% to about 2% by weight of amphoteric polymer;
(d) from about 0.15% to about 5% by weight of acrylates copolymer;
(e) from about 0.01% to about 4% by weight of silicone;
(f) from about 0.01% to about 4% by weight of alcohol ester; and
wherein the composition is clear in appearance, free of cationic polymer and has a pH of greater than about 5.

18. (canceled)

19. A process for cleansing and conditioning hair comprising contacting the hair with the composition of claim 17.
